(19) United States
(12) Patent Application Publication
(10) Pub. No.: US 2009/0004294 A1
(43) Pub. Date: Jan. 1, 2009

(54) PERSONAL LUBRICANT FORMULATIONS COMPRISING COLLOIDAL METALS AND METHODS OF USE

(75) Inventors: Joel Margulies, Lascassas, TN (US); Terry Jacobs, Marina Del Rey, CA (US)

Correspondence Address: KNOBBE MARTENS OLSON & BEAR LLP, 2040 MAIN STREET, FOURTEENTH FLOOR, IRVINE, CA, 92614, US

(73) Assignee: PharmWest, Inc., Marina Del Rey, CA (US)

(21) Appl. No.: 11/936,696

(22) Filed: Nov. 7, 2007

Related U.S. Application Data
(60) Provisional application No. 60/857,270, filed on Nov. 7, 2006.

(51) Int. Cl. A61K 33/24; A61P 31/12

(52) U.S. Cl. 424/649

(57) ABSTRACT

Lubricant compositions and methods are disclosed for aiding and enhancing massage or sexual activity, treating and preventing sexually transmitted diseases, and preventing pregnancy by administering a colloidal metal containing composition. Colloidal metal containing compositions that include metal flakes, sperm-function inhibitors, antimicrobial agents, analgesics, plant extracts, anti-oxidizing agents, and anti-inflammatory agents are also provided and may advantageously be used to in the methods of the invention.

This application claims the priority benefit to U.S. Provisional No. 60/857,270 filed Nov. 7, 2006, the entirety of which is hereby incorporated by reference herein.

### BACKGROUND OF THE INVENTION

1. Field of the Invention

The present invention generally relates to personal lubricant compositions comprising colloidal metals and optionally including metal flakes, and their applications for massage, aiding and enhancing sexual activity, treating and preventing transmission of sexually transmitted diseases (STDs), and contraception.

2. Description of the Related Art

It is believed that physical contact through, for example, sexual activity or massage, contributes to the emotional and physical well-being of human relationships. With respect to sexual activity, oftentimes individuals or sexual partners may experience diminished sexual response such as, for example, pain or discomfort during intercourse from lack of lubrication and/or diminished blood flow to the genital area.

Moreover, for the aging female population, a significant number of menopausal women suffer from various physiological changes that can affect sexual function. These include shrinking and thinning of the vagina and labia. The vagina becomes narrower and the natural swelling and lubrication that occurs during the arousal phase of the intercourse can take longer. This can cause difficult, painful, and/or frustrating sexual activity that affects both the physical and emotional well-being of individuals and partners.

Additionally, clitoral vascular engorgement plays an important role in female sexual arousal and overall sexual satisfaction. Sexual arousal results in smooth muscle relaxation and arterial vasodilation within the clitoris. The resultant increase in blood flow leads to tumescence of the glans clitoris and increased sexual arousal. A variety of diseases, such as arteriosclerosis and diabetes, may cause clitoral erectile insufficiency and reduced clitoral arterial flow. This, in turn, may lead to difficulty or inability to achieve clitoral tumescence, especially in women who suffer from Female Sexual Arousal Disorder (FSAD).

In addition to sexual activity, other avenues of physical contact have proven to show therapeutic physical and emotional benefits. One such area is that of massaging and massage therapy. Massage is one of the oldest forms of physical therapy in existence. From Swedish massage to Japanese shiatsu, massage has assumed many variations across many cultures. Massage is a system of stroking, pressing and kneading different areas of the body to relieve pain, and relax and stimulate the soft tissues (i.e. muscles, fascia, tendons, and ligaments). Massage can improve blood circulation and assists the lymphatic system, improving the elimination of waste throughout the body. Regular massage may also alter chronic muscle tension through effects on autonomic reflexes (i.e. muscle spindles and Golgi tendon organ) and by the release of restrictions and adhesions within the fascia surrounding the muscle. Despite the many advantages that come with massage, the physical contact necessary for massage inevitably carries the risk of spreading illness and disease. These transmissions can include transfer of bacterial, viral, or fungal infections between a massage therapist and a patient and/or massage partners.

In addition to these impediments, the increasing prevalence of STDs, such as syphilis, gonorrhea, herpes, genital warts, and Chlamydia, is a serious public health problem affecting physical contact, sexual activity, and relationships between individuals. For example, AIDS, or Acquired Immune Deficiency Syndrome, is a fatal disease that presently infects millions of people worldwide. Great efforts have been made to control and treat individuals with AIDS; however, to date, there is no cure and prevention is still a major means of combating the spread of the disease.

Furthermore, although there is always a risk of infection with unprotected sex, for large numbers of individuals, the risk of infection is even greater because of the physiological factors described above. For example, as described, some individuals such as menopausal women, have difficulty maintaining sufficient lubrication to facilitate intercourse comfortably. In such cases, vaginal dryness and the lack of lubrication can cause inflammation, chaffing, tearing, and/or bleeding of mucosal membranes. Irritation, open wounds, cuts, and sores all increase the likelihood of STD transmission and infection.

Contraceptive agents have previously been used in conjunction with lubricating compositions. Spermicides such as nonoxynol-9 and benzalkonium chloride have been used effectively as active ingredients in contraceptive compositions for many years. For many individuals, it is desirable to conveniently administer contraceptive, antimicrobial, and lubricating agents in a single, rather than multiple compositions.

For these reasons, there is a continuing need to develop a formulation that is harmless to skin and mucosal membranes, effective against infectious pathogens such as Hepatitis viruses, HIV, Herpes simplex virus (HSV), Neisseria gonorrhoeae (NG), and Chlamydia trachomatis (CT), and at the same time aiding and/or enhancing massage or sexual activity, while providing contraceptive protection.

### SUMMARY OF THE INVENTION

In one aspect of the invention, lubricating compositions are provided. A lubricating composition, according to the present invention, preferably comprises a colloidal metal and suitable lubricating carrier. In some embodiments, the colloidal metal is colloidal gold. Preferably, the colloidal metal has a particle size from about 1-200 nm. In additional embodiments, the lubricating compositions comprise colloidal metal and metal flakes. In some embodiments, the metal flakes have a particle size from about 1-5 mm. In further embodiments, the colloidal metal comprises from about 0.001-25 percent by weight of the composition. Preferably, the lubricating compositions comprise colloidal gold and gold flakes.

In some embodiments, the compositions further comprise at least one additional active agent. In other embodiments, the additional active agents are selected from the group consisting of antimicrobials, anti-inflammatory agents, analgesics, contraceptive agents, vasoactive agents, antioxidants, and plant extracts. In some embodiments, the contraceptive agent is a sperm-function inhibitor, such as a spermicidal agent.

In further embodiments, the lubricating compositions comprise a suitable lubricating carrier selected from the group consisting of water-based, silicone-based, petroleum-based, and natural oil-based formulations. In other embodiments, the suitable lubricating carrier is acceptable for administration to the skin or mucosa. In further embodiments, the suitable lubricating carrier comprises acceptable excipients for a massage formulation.

In additional embodiments, the lubricating compositions further comprise inactive components. These inactive components may be selected from the group consisting of sensory additives, massage oils, essential oils, preservatives, humectants, thickening agents, moisturizing agents, acidic or alkaline substances, buffering agents, anti-crystallization agents, and lubricating agents. In some embodiments, the sensory additives are selected from the group consisting of color, smell, taste, warming, cooling, and variable temperature agents.

In another aspect of the invention, methods for massaging are provided. In some embodiments, the invention provides a method of massaging by administering to an animal in need thereof, an amount of the lubricating composition sufficient to allow massaging activity.

In another aspect of the invention, methods for aiding and enhancing sexual activity are provided. In some embodiments, the invention provides a method of aiding and/or enhancing sexual activity by administering to an animal in need thereof, an amount of the lubricating composition sufficient to enhance or aid sexual activity. In other preferable embodiments, the method is administered to a human.

In another aspect of the invention, methods for treating and/or preventing transmission of sexually transmitted diseases are provided. In some embodiments, the invention provides a method for treating and preventing transmission of sexually transmitted diseases by administering to an animal in need thereof, an amount of the lubricating composition sufficient to treat or prevent the transmission of sexually transmitted disease. In other preferable embodiments, the method is administered to a human. In some embodiments, the sexually transmitted disease is selected from the group consisting of Chancroid, Chlamydia, Gonorrhea, Hepatitis, Herpes, HIV, AIDS, Human Papilloma virus, and Syphilis.

In another aspect of the invention, methods for contraception are provided. In some embodiments, the invention provides a method for contraception by administering to an animal in need thereof, an amount of the lubricating composition sufficient to provide contraceptive activity. In other preferable embodiments, the method is administered to a human.

In another aspect of the invention, methods for simultaneously aiding and/or enhancing sexual activity, treating and preventing transmission of sexually transmitted diseases, and contraception are provided. In some embodiments, the present invention provides for methods of aiding and/or enhancing sexual activity and treating and/or preventing transmission of STDs by administering to an animal in need thereof, an amount of the composition sufficient to provide dual activity. In other embodiments, the present invention provides for methods of aiding and/or enhancing sexual activity and contraception by administering to an animal in need thereof, an amount of the composition sufficient to provide dual activity. In further embodiments, the invention provides for methods of treating and/or preventing transmission of STDs and contraception by administering to an animal in need thereof, an amount of the composition sufficient to provide dual activity. In still other embodiments, the invention provides for methods of simultaneously aiding and/or enhancing sexual activity, treating and/or preventing transmission of STDs, and contraception by administering to an animal in need thereof, an amount of the composition sufficient to provide at least dual activity. In other preferable embodiments, the methods described above are administered to a human.

In another aspect, the present invention provides lubricating compositions consisting essentially of water, glycerin, glyceryl acrylate/acrylic acid copolymer, propylene glycol, hydroxyethylcellulose, carbomer, colloidal gold, niacin, arginine, panax ginseng extract, cinnamon essential oil, polyquaternium-7, 24K gold leaf, sodium hydroxide, methylparaben, propylparaben, and diazolidinyl urea.

In yet another aspect, the present invention provides for lubricating compositions consisting essentially of cyclopentasiloxane, dimethicone/vinyl dimethicone crosspolymer, arginine, colloidal gold, and 24K gold leaf.

### DETAILED DESCRIPTION OF THE PREFERRED EMBODIMENT

The compositions and methods of the present invention relate to aiding and enhancing massage and sexual activity, treating and preventing STD transmission, and contraception.

The phrase, “sexually transmitted diseases (STDs),” includes any disease that is capable of being transmitted in the course of sexual contact, whether or not the genital organs are affected by the resulting pathology.

### Therapeutic and Prophylactic Agents

Antimicrobial Agents

In the context of the present invention, an “antimicrobial agent” generally refers to a material that has sufficient antimicrobial activity to inhibit transmission of bacterial, viral, or fungal infections. In certain embodiments, an antimicrobial material has sufficient antimicrobial activity to provide beneficial therapeutic effect.

Although numerous organic compounds are known to possess potent contact-mediated antimicrobial actions (e.g., alcohol), such compounds are poorly suited for long-term protection. In accordance with preferred aspects of the present invention, inorganic metal containing materials are used as an antimicrobial agent, such as metals having antimicrobial properties. Silver, gold, copper, zinc, tin, mercury, lead, iron, cobalt, nickel, manganese, arsenic, antimony, bismuth, barium, cadmium and chromium have been known for a long time as metals which exhibit antifungal, anti-algal and antibacterial activities. For example, silver has been widely used in the form of aqueous silver nitrate solution as bactericidal and/or disinfectant solutions. Additionally, copper is believed to exhibit strong anti-fungal and antibacterial properties. However, some of the above-mentioned antimicrobial metals have been found to be toxic for humans. Furthermore, some of the toxic metals also have various practical and regulatory limitations in methods of use, storage and disposal. Consequently, their use as antimicrobial agents has been limited.

Recently, applicant has found that relatively small concentrations of colloidal antimicrobial metals, gold in particular, when combined with a non-toxic, suitable carrier, is sufficient to provide desired antimicrobial activity without risk of toxic effects for humans.

Composite materials, comprising a colloidal antimicrobial metal in combination with a suitable lubricating carrier are preferred in accordance with the present invention. Gold-based inorganic antimicrobial agents are most preferred. These inorganic antimicrobial agents cause less skin irritation and offer much improved longevity, when compared with typical volatile organic agents.

Anti-Inflammatory Agents

In the context of the present invention, an “anti-inflammatory agent” generally refers to a material that has sufficient anti-inflammatory activity to reduce inflammation. In certain embodiments, an anti-inflammatory agent has sufficient anti-inflammatory activity to provide a beneficial therapeutic effect.

In accordance with preferred aspects of the present invention, inorganic metal containing materials are used as anti-inflammatory agents. For example, gold, copper, and silver have been known as metals which exhibit anti-inflammatory effects. In particular, gold has been widely used in the form of gold salts, such as disodium aurothiomalate, for the treatment of inflammation in rheumatoid arthritic conditions.

In some embodiments, a colloidal anti-inflammatory metal, such as colloidal gold, is combined with a non-toxic, suitable carrier, sufficient to provide the desired anti-inflammatory activity without risk of toxic effects for humans.

Anti-Oxidizing Agents

Antioxidants inhibit inter alia lipid peroxidation, which may thereby prevent and minimize damage to physiological systems. Such damage can contribute to accelerated aging or formation of cancerous tissues. Natural anti-oxidizing enzymes such as superoxide dismutase are effective antioxidants in mitigating and preventing the damage caused by free radicals associated with oxidation and lipid peroxidation. Specifically, superoxide dismutase converts free radical oxygen species (ROS) into water and oxygen molecules. Although an effective antioxidant, problematically, natural superoxide dismutase levels decrease with aging.

As a result of intensive investigation into preventing oxidative damage, the present inventors have found that metals, such as gold and copper, exhibit anti-oxidizing properties. Without wishing to be bound by any theory, it is believed that in some embodiments of the present invention, colloidal gold is an effective catalyst for the elimination of free radicals such as ROS. (Esumi et. al., Langmuir, 2004, 20, 2536-2538) Accordingly, the colloidal gold containing compositions of the invention provide anti-oxidizing benefits for the skin surface or mucosal membrane, which prevents and minimizes the effects of radical damage.

Colloidal Metal and Metal Flakes

Some embodiments of the invention provide for a composition comprising an antimicrobial colloidal metal dispersion. Preferably, the colloidal metal has a particle size ranging about 1-200 nanometers (nm). In some embodiments, the colloidal metal has a particle size less than about 100 angstroms. In some embodiments, the particle size is less than about 25 angstroms. In some preferred embodiments, the particle size is about 1.5-5 nm. In some embodiments, the metal's antimicrobial, anti-inflammatory, and/or anti-oxidizing properties are dependent on the particle size of the colloidal metal. The smaller particle sizes are better configured for permeation through the mucosal lining and underlying cellular membranes. In other embodiments, the colloidal metal comprises about 0.001 to 25 percent by weight of lubricating composition. In further embodiments, the pH of the colloidal formulation is from about 4.5-6. Additionally, some formulations, according to the present invention, may exhibit coloration. For example, in some embodiments, a colloidal gold containing lubricating composition shows a purple tint. Of course, the lubricant formulations may be produced in any color, by adding coloring agents well known to those of skill in the art.

The preparation of colloidal metal can be accomplished by various methods. For example, methods for preparing colloidal gold include citrate reduction whereby gold nanoparticles are produced by reduction of gold(III) derivatives, such as HAuCl₄ in water, or the Burst-Schiffrin Method as described in Daniel et. al., Chem. Rev. 2004, 104, 293-346, which is incorporated by reference herein.

In some embodiments, the colloidal metal consists of particles or clusters of pure metal suspended in pure and de-mineralized water with no ionic or electric charge. In other embodiments, the metal particles are in constant motion, exhibiting Brownian motion. Without being bound to any theory, it is believed that this motion results in charged metal particles. Consequently, in some embodiments, the like-charged metal particles exert repulsive forces on each other, which further improves uniform distribution.

In some embodiments, the compositions of this invention may contain both colloidal metals and solid metal flakes, preferably made of gold or other noble metal. In further embodiments, the metal particles (e.g., gold) are in the form of flakes, leaves, powder or shavings. Although the terms “flakes,” “particles,” “leaves,” “powder,” and/or “shavings,” are used to describe the metal's appearance and form, these labels are for convenience and do not indicate the specific shape, thickness, and/or other physical properties.

For some transparent or translucent embodiments, the metal flakes will typically be seen to settle to the bottom of the container, but when the composition is shaken, the metal flakes will float throughout the medium and will eventually settle by gravitational forces. It other embodiments, the medium is of such viscosity so as to maintain a more uniformly distributed suspension of metal flakes throughout the composition.

In further embodiments, noble metal flakes, such as gold, will be clearly visible in a transparent or essentially transparent medium and will create a sense of luxury and opulence in the user. In still further embodiments, the metal flakes are present in sufficient amounts to provide an aesthetically distinct metal-sprinkled appearance. Yet at the same time, the metal particles will not be noticeable to any great degree when the product is applied in use.

The size of the metal particles can vary widely. In some embodiments, the metal flakes are of a size, shape, texture, thickness, etc. so that when the composition is administered, the particles will disappear or be essentially invisible to the unaided eye. In some embodiments, the size of the metal flakes range from about 1-5 millimeters (mm).

In some embodiments, the colloidal metal and/or metal flakes comprise alloys containing at least two metals. In some embodiments, the alloys include those with about 50% Ag, about 50% Au, about 50% Cu, about 60% Au, about 20% Ag, or about 20% Cu. Furthermore, the metals described in the present invention may be of either crystalline and/or amorphous structure. In some embodiments, the crystalline and amorphous metal particles may be separated.

Preferably, the lubricating compositions of the present invention comprise a colloidal noble metal with at least one of the antimicrobial, anti-inflammatory, and anti-oxidizing properties. In preferable embodiments, colloidal gold is present in such amounts so as to have an antimicrobial, anti-inflammatory, and anti-oxidizing effect on the applied surface. In additional preferred embodiments, gold flakes are visibly dispersed and suspended in the lubricating composition. In further preferred embodiments, the lubricating composition comprises copper, either in colloidal form or both colloidal and flake form.

Additional Active Agents

Optionally, in some embodiments, the composition may comprise of additional active agents. In some embodiments, the composition comprises at least one additional active agent. As defined herein, an “active agent” includes an agent that exhibits antimicrobial activity, or anti-STD pathogen activity, antibacterial activity, antiviral activity, antifungal activity, antiparasitic activity, antiprotozoal activity, anti-inflammatory activity, analgesic activity, contraceptive activity, and vasoactive activity. The active agent may have multiple functions, such as acting as a spermacide and an antimicrobial agent. Although described with specific examples, one of skill in the art will recognize that the active agents described herein are illustrative rather than exhaustive of applicable additional active agents.

In some embodiments, the total amount of additional active agent(s), or mixtures thereof, will typically range from about 0.1 to 50 weight percent of the compositions according the present invention. Preferably, the amount of additional active agent employed will be that amount necessary to achieve the desired activity and results. Appropriate amounts can be determined by those skilled in the art.

Antimicrobial—Accordingly, in some embodiments, the antimicrobial agent may be an antibacterial, antifungal, antiviral, antiparasitic, and an antiprotozoal agent. The antimicrobial agent is preferably active against infectious diseases, such as STDs. Examples of microorganisms that cause such diseases (and the diseases caused by such microorganisms) include Neisseria gonorrhoeae (gonorrhea); Chlamydia trachomatis (chlamydia, lymphogranuloma venereum); Treponema pallidum (syphilis); Haemophilus ducreyi (chancroid); Calymmatobacterium granulomatis (donovanosis), Mycoplasma genitalium, Ureaplasma urealyticum (mycoplasmas); human immunodeficiency virus HIV-1 and HIV-2 (HIV, AIDS); HTLV-1 (T-lymphotrophic virus type 1); herpes simplex virus type 1 and type 2 (HSV-1 and HSV-2); Epstein-Barr virus; cytomegalovirus; varicella-zoster virus; human papilloma viruses (genital warts); hepatitis A virus, hepatitis B virus (viral hepatitis); Trichomona vaginalis (trichomoniasis); and yeasts, such as Candida albicans (vulvovaginal candidiasis).

The antimicrobial agent may also be active against other diseases that are transmitted by contact with bodily fluids (e.g., hepatitis) that may also be transmissible by sexual contact and are capable of being prevented by administration of the compositions according to this invention.

Suitable antiviral agents include, for example, virus-inactivating agents and nucleotide or nucleoside analogs, such as tenofovir, acyclovir, amantadine, didanosine, foscarnet, ganciclovir, ribavirin, vidarabine, zalcitabine, and zidovudine. Further antiviral agents that may be used include non-nucleoside reverse transcriptase inhibitors, such as UC-781 (thiocarboxanilide), pyridinones, TIBO, nevaripine, delavirdine, calanolide A, capravirine and efavirenz.

Other antiviral agents that may be used in combination with colloidal metals are those in the category of HIV entry blockers, such as cyanovirin-N, clyclodextrins, carregeenans, sulfated or sulfonated polymers, mandelic acid condensation polymers, monoclonal antibodies, chemokine receptor antagonists, and fusion inhibitors.

Suitable antibacterial agents include antibiotics, such as aminoglycosides, cephalosporins; macrolides, including erythromycins, penicillins, including natural penicillins, penicillinase-resistant penicillins, aminopenicillins, extended spectrum penicillins; sulfonamides, tetracyclines, fluoroquinolones, metronidazole and urinary tract antiseptics.

Suitable antifungal agents include amphotericin B, nystatin, griseofulvin, flucytosine, fluconazole, potassium iodide, intraconazole, clortrimazole, miconazole, ketoconazole, and tolnaftate.

Suitable antiprotozoal agents include antimalarial agents, such as chloroquine, primaquine, pyrimethamine, quinine, fansidar, and mefloquine; amebicides, such as dioloxamide, emetine, iodoquinol, metronidazole, paromomycine and quinacrine; pentamidine isethionate, atovaquone, and eflornithine.

Anti-inflammatory Agents—In addition to antimicrobial agents, some embodiments contain additional anti-inflammatory agents such as additional anti-inflammatory metals, nonsteroidal anti-inflammatory agents, and/or natural anti-inflammatory substances, for example, aloe vera, allantoin, or chamomile.

Analgesics—Further embodiments of the compositions of this invention are compositions that include analgesics. The analgesics may include, but are not limited to, methyl salicylate, methyl nicotinate, capsicum, benzocain, lidocaine, dibucaine, benzyl alcohol, camphor, resorcinol, menthol, and diphenylhydramine hydrochloride and the like.

Contraceptive Agents—In some embodiments, the active agent may also be a contraceptive and/or sperm-function inhibitor with the ability to inhibit the function of sperm, to inhibit fertilization of an egg by sperm, and/or to otherwise prevent pregnancy, such as by killing and/or functionally inactivating sperm or by other effects on the activity of the sperm. In some embodiments, the additional sperm-function inhibitor is a spermicide useful in accordance with the present invention. Examples of such spermicidal agents include benzalkonium chloride, octoxynol-9, ricinoleic acid, phenol mercuric acetates, and nonoxynol-9, etc.

Vasoactive Agents—In some embodiments, the lubricating composition may contain a vasoactive agent. A vasoactive agent is interpreted to be a substance or material that works to affect blood flow in vascular tissue. These agents include vasodilators that improve blood flow and circulation. In some embodiments, the present invention comprises vasodilators that promote circulation and improve blood flow to the vaginal and clitoral area. In other embodiments, the presence of a vasodilator contributes to vaginal and or clitoral engorgement, lubrication, and sexual arousal.

In further embodiments, the present invention provides compositions for aiding vasodilation of the penile arterial blood vessel. The vasoactive agent may comprise any known erectogenic compound which, on absorption through the skin or mucosa, locally enhances blood flow. Such compounds may include natural, semi-synthetic, and synthetic prostaglandins, nitrates, long and short acting alpha-adrenoceptor blockers, ergot alkaloids, and anti-hypertensives.

Vasoactive agents can be used alone or in combination and, optionally, together with skin penetration enhancers such as azone and dimethylsulfoxide (DMSO). Esters of prostaglandins, such as the methyl and ethyl esters, can also be used.

Antioxidants—Some embodiments may also include antioxidants such as superoxide dismutase, curcumin, camosin, polyphenolics, catalase, glutathione peroxidase, oligomeric proanthocyanidins, bioflavonoids, leuco anthocyanin, anthocyanidin, alpha-lipoic acid, coenzyme Q10, selenium, and vitamin C.

Plant Extracts—Compositions of the present invention may also include plant extracts, for example, aloe, witch hazel, chamomile, licorice extract, and hydrogenated soy oil.

Inactive Components

In addition to the active agents, some embodiments comprise inactive components such as sensory additives, preservatives, humectants, and other suitable carrier components. An “inactive component” refers herein to relatively biologically inactive components.

Sensory Additives—A “sensory additive” as defined herein refers to a substance, material, agent, compound, etc., that affects, modifies, is associated with, and/or contributes to sensory perception. These additives include color, flavor, scent, warming, cooling, and/or variable temperature agents.

In some embodiments, color agents, such as glitter, dyes, colorants, and/or pigments, may be added to the lubricating compositions. In other embodiments, flavoring or scenting agents such as peppermint oil may be added to the composition to impart smell and/or taste. In further embodiments, a temperature sensory additive agent may be added to generate warmth, coolness, or otherwise affect the perception of temperature. Additionally, some embodiments provide that when mixed with water or in contact with skin or mucosa, the compositions increase/decrease in temperature, generate warmth, and/or exhibit a variable decrease and increase of warmth and coolness. In further embodiments, the sensory additive results in a soothing and/or stimulating effect on the tissues to which these compositions are applied.

In some embodiments, the lubricating compositions according to the invention can be used as massage oils. In addition, some embodiments provide lubricating compositions that can serve as dual lubricating and massage compositions. In other embodiments, massage oils and/or essential oils are added to the lubricating composition.

These massage and/or essential oils can include seed oils, oils of mandarin, (Citrus reticulata var. mandarin), sage (Salvia officinalis), geranium rose (Pela graveolens xasperium), palmarosa (Cynbopogon martini), nutmeg (Myristica frangrans), rosewood (Aniba roseaodora), cedarwood (Juniperus virginiana), patchouli (Pogostemon cablin), cardamom (Elettaria cardamomum), vetiver (Vetivertia zizanioides), orange (Citru sinensis L. osbeck), sandalwood (Santalum album), clary sage (Salvia sciarea), rose (Rosa centifolia), jasmine, (Jasminum grandiflorum), yarrow (Achillea millefolium), tanacetum (Tanacetum annuum), ylang ylang (Cananga odorata), rosemary (Rosmarinus officinalis), birch (Betula lenta), grapefruit (Citrus paradisi), cypress (Cupressus sempervirens), peppermint (Mentha piperita), bay laurel (Laurus nobilis), black pepper (Piper nigrum), ginger root (Zingiber officinale), juniper berry (Juniperus communis), lemongrass (Cymbopogon flexuosus), and wintergreen (Gaultheria procumbens).

Carrier Components—As noted above, the compositions and methods of the present invention comprise suitable lubricating carriers. In some embodiments these carriers are selected from water-based, petroleum-based, silicone-based, or natural oil-based formulation.

By way of example, a water-based formulation may include water, a thickening agent, a lubricating agent, and preservatives. For some embodiments, a thickening agent, such as cellulose or a chemically treated derivative of cellulose, acacia, agar, alginate, carrageenan, gum tragacanth, xanthan gum, collagen, carboxypolymethylene, glyceryl monostearate, polyvinylpyrrolidone, and/or polyacrylamide may be used.

As used herein, “lubricating agent” refers to a component which is incorporated into a lubricating composition for the purpose of reducing friction during sexual activity. Although any liquid (including water) sometimes functions as a “lubricant” in the broadest sense of the word, a preferred lubricating agent (1) feels slippery when rubbed between skin or mucosal surfaces; (2) when applied to skin or mucosal surfaces, spreads smoothly and evenly across the contacted area; (3) remains in contact with the skin or mucosal surfaces; and (4) does not evaporate or become sticky quickly. Additionally, preferred lubricating agents are gradually broken down into innocuous substances in the body or they are of a nature that allows them to be secreted by the contacted skin or mucosa and/or washed cleanly from the site of contact.

Several lubricating agents which are used in commercially available lubricants satisfy these criteria, including glycerin (also called glycerine, glycerol, 1,2,3-propanetriol, and trihydroxypropane) and certain types of polyethylene glycol (PEG), such as PEG 200 or PEG 400 (the numbers indicate different molecular weight averages). Various other polymers (such as polypropylene glycol, polyisobutene, and polyoxyethylene) and certain naturally-occurring compounds (such as behenic acid, derived from various types of seeds and animal fats) and their derivatives (such as behenyl alcohol) are also used as lubricants in cosmetics and other formulations that contact the skin or mucosa. In addition, some sugar-alcohols such as sorbitol, and some silicon compounds such as polydimethylsiloxane, are also used as lubricating agents.

In some embodiments, the lubricating compositions may further include suitable preservatives well known in the art, including, for example, methyl paraben, propyl paraben, butyl paraben, benzoic acid and benzyl alcohol.

The lubricating compositions described may further include diluents, fillers, binding agents, moisturizing agents, emollients, anti-crystallization agents, alkaline or acidic or buffering agents to maintain the proper pH, and other components known to those skilled in the art. For example, solid, liquid or a mixture of solid and liquid suitable carriers, diluents, vehicles, or excipients may be employed in the lubricating compositions.

### Methods of the Present Invention

In preferred embodiments, the present invention also relates to methods of administering any of the mentioned compositions for aiding and enhancing massage or sexual activity, treating and preventing the transmission of STDs, and contraception.

By “administering” or “application,” it is meant that a composition is delivered to the subject in such a way that it can achieve the desired purpose, e.g., aiding or enhancing sexual activity. Compositions of the present invention can be administered to any suitable subject, including animals, humans, and other organisms.

In some embodiments, compositions of the present invention canaid, facilitate, and/or enhance massage and sexual activity by reducing friction experienced on the skin or mucosal surface. In some embodiments, the compositions and methods of the present invention provide for administering the lubricating compositions onto the skin or mucosal surface of an animal or human. In other embodiments, the lubricating compositions are applied in sufficient amounts to provide varying degrees of resistance between at least two surfaces such as the surface of a massage therapist's hands and a patient's body. In further embodiments, the present invention facilitates massage by administering an amount of the lubricating compositions to provide the sensation of temperature change (e.g., warming, cooling, variable temperature perception). In yet further embodiments, the lubricating compositions are administered in sufficient amounts to improve blood flow and circulation at the site of application.

Some embodiments of the compositions and methods of this invention provide for administering the compositions of the present invention onto a mucosal membrane such as a buccal, vaginal, or rectal cavity. In other embodiments, the compositions of the present invention are applied to perianal areas and/or the lining of the anus. In further embodiments, the method of this invention provides for the application of the lubricating compositions described above to genital areas and/or genitalia. In still further embodiments, the method provides for the application of the lubricating compositions described to the vulva or penis.

In some embodiments, compositions of the present invention can facilitate, aid, and enhance sexual activity by aiding, for example, arousal and/or orgasm, by improving blood flow, vasodilation, and sensory input and perception. In some embodiments, a lubricating composition containing a vasodilator, a warming agent, a flavoring, and a scenting agent can be used to attain, aid, maintain, and/or enhance sexual interest and sexual excitement for individuals suffering from sexual arousal disorders and conditions such as absence of libido, decreased clitoral or labial engorgement, and/or absence of natural vaginal lubrication. In further embodiments, the lubricating composition can be used as a clitoral stimulant.

Generally, “mucosa” and “mucosal membrane” refer to the lubricated lining of the mouth or vagina or any membrane or tissue containing a mucous secreting gland.

By the terms, “enhance” and “aid,” it is generally meant that administration of the composition increases the subject's satisfaction with the physical or sexual activity as compared to the activity when in the absence of the composition. This includes, e.g., enhancement of mucosal wetness, relaxation, warmth, engorgement, sensitivity, sensation, tingling, arousal, and orgasm.

In other embodiments, the methods of the present invention relate to methods of treating and preventing the transmission of STDs. The lubricating compositions of the present invention, according to the methods described herein, are administered or otherwise applied to a site of infection or likely infection. The site of infection may be one where an infection is already present (an actual site of infection) or where an infection is likely to occur (a potential site of infection in or on an uninfected individual). The amount of administered lubricating composition may vary depending upon factors such as the STD-causing microorganism intended to be inhibited.

Accordingly, the lubricating compositions may be administered or applied to the vulva, including the vaginal cavity, the penis and the ano-rectal and buccal cavities by contacting the skin or mucosal membranes of the intended site or surrounding the intended site. The mucosal or skin surface may further include the perianal, and the lining of the anus. For example, in the case of inhibiting STD-infections, the lubricating compositions of the present invention may be administered by being contacted with any potential or actual sites of infection, including the vaginal, ano-rectal or buccal cavities to prevent STD infection during intimate activity. The lubricating compositions can also be applied to external genitalia and internal mucosal surfaces to reduce microtrauma resulting from inadequate lubrication and to prevent transmission of viable STD pathogens through traumatized, diseased or healthy skin or mucosa.

“Preventing” and “treating” as referenced in the methods of the present invention are intended to mean at least the reduction in incidence or prevalence of the occurrence of the specified activity relative to an untreated individual. These terms further refer to reduced transmission of STD infection as a result of treatment of the individual prior to, or immediately after, intimate contact relative to untreated individuals.

In some embodiments, the present invention provides methods of contraception by indirectly or directly administering the lubricating composition to a subject in need thereof. This may be done, for example, by applying the lubricating composition directly onto or into the vaginal area or indirectly by applying the composition onto a condom or other device. In other embodiments, the methods of the present invention provide for administering an amount of the lubricating composition so as to decrease the possibility of sperm-egg fertilization, either by blocking entry of sperm into the egg, inhibiting sperm-fertilizing capabilities or by other methods. The amount of contraceptive substance, compound, or material may be readily determined by one of skill in the art.

In some embodiments, the present invention provides a method of using the lubricating composition to simultaneously aid and enhance sexual activity and treat and/or prevent transmission of STDs. Accordingly, these methods provide for administration of described lubricating composition to a skin or mucosal surface in sufficient amounts to dually aid and enhance sexual activity as well as treat and prevent STD transmission. In other embodiments, the present invention provides for methods of simultaneously aiding and/or enhancing sexual activity and providing contraceptive activity. In further embodiments, the present invention provides for methods of simultaneously treating and/or preventing STD transmission while also providing contraceptive activity. In still further embodiments, methods for simultaneously aiding and enhancing sexual activity, treating and preventing transmission of STDs, and contraception are provided.

The terms “simultaneous,” “simultaneously,” or “dually” do not require temporal identity, rather the terms refer to the use of the described lubricating compositions for multiple purposes. For example, a lubricating composition containing a spermicidal agent can dually aid and enhance sexual activity as well as provide a contraceptive effect. In this example, a lubricating composition of the present invention can provide both benefits through administration to skin or mucosal surfaces; however, the term “dually” does not require that the effects occur at the exact same time, even though they may.

As will be appreciated now, the novel compositions and methods of the present invention will provide extensive application for facilitating sexual or massage activity, protecting against STD infection, and contraception.

1. A lubricant composition, comprising a colloidal metal and a suitable lubricating carrier.

2. The composition according to claim 1, wherein the colloidal metal comprises gold.

3. The composition according to claim 1, further comprising metal flakes.

4. The composition according to claim 3, wherein the metal flakes comprise gold flakes.

5. The composition according to claim 1, wherein the colloidal metal comprises particles having a size from about 5-200 nm in a colloidal suspension.

6. The composition according to claim 1, wherein the metal flakes comprise particles having a size from about 1-5 mm.

7. The composition according to claim 1, wherein the colloidal metal comprises from about 0.001-25 percent by weight of the composition.

8. The composition according to claim 1, further comprising of at least one additional active agent.

9. The composition according to claim 8, wherein said at least one additional active agent is selected from the grout consisting of an antimicrobial agent, an analgesic, a contraceptive agent, an anti-inflammatory agent, a plant extract, a vasoactive agent and an antioxidant.

10-17. (canceled)

18. The composition according to claim 1, wherein the suitable carrier comprises acceptable excipients for skin and/or mucosal administration.

19. The composition according to claim 18, wherein the acceptable excipients are selected from one or more of the group consisting of a water-based formulation, a silicone-based formulation, a petroleum-based formulation and a natural oil-based formulation.

20-22. (canceled)

23. The composition according to claim 1, wherein the suitable carrier comprises acceptable excipients for a massage formulation.

24. The composition according to claim 1, further comprising inactive components.

25. The composition according to claim 24, wherein said inactive components are selected from the group consisting of sensory additives, massage oils, essential oils, preservatives, humectants, thickening agents, moisturizing agents, emollients, acidic or alkaline substances, buffering agents, anti-crystallization agents, and lubricating agents.

26. The composition according to claim 25, wherein said sensory additive is selected from the group consisting of color, smell, taste, warming, cooling, and variable temperature agents.

27. A method of massaging, comprising applying to a mucosal area or skin surface of an animal in need thereof, an amount of the composition of claim 1, wherein the amount is sufficient to facilitate massage activity.

28. A method of enhancing and aiding sexual activity, comprising applying to a mucosal or skin surface of an animal in need thereof, an amount of claim 1, wherein the amount is sufficient to enhance or aid sexual activity.

29. A method of treating and/or preventing sexually transmitted diseases, comprising applying to a mucosal or skin surface of an animal in need thereof, an amount of the composition of claim 1, wherein the amount is sufficient to treat or prevent the sexually transmitted disease.

30. The method according to claim 29, wherein the sexually transmitted disease is selected from the group comprising of Chancroid, Chlamydia, Gonorrhea, Hepatitis, Herpes, HIV, AIDS, genital warts, and Syphilis.

31. A method of aiding contraception, comprising applying to a mucosal or skin surface of an animal in need thereof, an amount of the composition of claim 1, wherein the amount is sufficient to provide contraceptive activity.

32. A method of simultaneously aiding and/or enhancing sexual activity and treating and preventing a sexually transmitted disease, comprising applying to a mucosal or skin surface of an animal in need thereof, an amount of the composition of claim 1, wherein the amount is sufficient to simultaneously aid and/or enhance sexual activity and treat and/or prevent the sexually transmitted disease.

33. A method of simultaneously aiding and/or enhancing sexual activity and providing a contraceptive effect, comprising applying to a mucosal or skin surface of an animal in need thereof, an amount of the composition of claim 1, wherein the amount is sufficient to simultaneously aid and/or enhance sexual activity and provide the contraceptive effect.

34. A method of simultaneously treating and preventing a sexually transmitted disease and providing a contraceptive effect, comprising applying to a mucosal or skin surface of an animal in need thereof, an amount of the composition of claim 1, wherein the amount is sufficient to simultaneously treat and/or prevent the sexually transmitted disease and provide the contraceptive effect.

35. A method of simultaneously aiding and/or enhancing sexual activity, treating and/or preventing a sexually transmitted disease, and providing a contraceptive effect, applying to a mucosal or skin surface of an animal in need thereof, an amount of the composition of claim 1, wherein the amount is sufficient to simultaneously aid and/or enhance sexual activity, treat and/or prevent the sexually transmitted disease, and provide the contraceptive effect.

36. A lubricant composition, consisting essentially of water, glycerin, glyceryl acrylate/acrylic acid copolymer, propylene glycol, hydroxyethylcellulose, carbomer, colloidal gold, niacin, arginine, panax ginseng extract, cinnamon essential oil, polyquaternium-7, 24K gold leaf, sodium hydroxide, methylparaben, propylparaben, and diazolidinyl urea.

37. A lubricant composition, consisting essentially of cyclopentasiloxane, dimethicone/vinyl dimethicone crosspolymer, arginine, colloidal gold, and 24K gold leaf.
